(19) United States
(12) Patent Application Publication
(10) Pub. No.: US 2024/0044892 A1
(43) Pub. Date: Feb. 8, 2024

(54) ML-ASSISTED LYME DISEASE MICROARRAY ASSAY

(71) Applicant: InBios International, Inc., Seattle, WA (US)

(72) Inventor: James William Needham, Seattle, WA (US)

(21) Appl. No.: 18/365,831

(22) Filed: Aug. 4, 2023

Related U.S. Application Data
(60) Provisional application No. 63/480,890, filed on Jan. 20, 2023.
(60) Provisional application No. 63/395,266, filed on Aug. 4, 2022.

(51) Int. Cl. G01N 33/569; G01N 33/543

(52) U.S. Cl. CPC G01N 33/56911; G01N 33/54386; G01N 2333/195; G01N 2469/20

(57) ABSTRACT

The present disclosure relates to a machine learning (ML)-implemented assay for Lyme disease that detects antibodies for multiple Lyme disease associated antigens, as well as kits and systems for implementation of such an assay.

### PRIORITY INFORMATION

The present application claims priority under 35 U.S.C. § 119(e) to U.S. Provisional Patent Application Ser. No. 63/395,266, filed Aug. 4, 2022, and U.S. Provisional Patent Application Ser. No. 63/480,890, filed Jan. 20, 2023, both titled “ML-ASSISTED LYME DISEASE MICROARRAY ASSAY,” both of which are incorporated by reference herein in their entities.

### TECHNICAL FIELD

The present disclosure relates to microarray assays that can detect IgM and IgG antibodies against a plurality of antigens associated with Lyme disease, and that uses machine learning (ML)-assisted methods to determine if IgG and IgM binding to the plurality of antigens is indicative of Lyme disease.

### BACKGROUND

Lyme disease is an important and costly emerging disease in the United States. Lyme disease is the most prevalent vector borne disease in the US and is a growing health concern the incidence of which has doubled over the last 20 years to approximately 476,000 patients being treated for Lyme disease every year. Though originally endemic to the northeast and upper mid-west, Lyme disease has expanded its habitat during this time, spreading to non-endemic regions of the US.

Lyme disease in the US is caused predominantly by the spirochete Borrelia burgdorferi sensu lato and spread via the bite of a hard bodied tick, Ixodes Scapularis. Early symptoms of Lyme disease may appear within an average of 7 days after a tick bite and include fever, chills, headache, fatigue, muscle and joint aches, and swollen lymph nodes. The primary symptom of acute stage Lyme disease is a characteristic bulls-eye skin rash called Erythema Migrans (EM) which when detected, is considered confirmatory. Lyme disease is effectively treated with antibiotics, but, in about 30% of cases, the rash may not be noticed or detectable, leading to undiagnosed disease which, when left untreated, can progress to serious neurological, carditis and rheumatoid manifestations in a matter of weeks or months. Approximately 10-20% of patients not treated early in Lyme disease will develop chronic complications such as fatigue, musculoskeletal weakness and neurocognitive disorders such as memory loss. Thus, undiagnosed Lyme disease can have lifelong debilitating consequences.

Early and late Lyme disease symptoms and the uncertainties associated with diagnosis of Lyme disease cause a significant economic burden on the health system as well. The Johns Hopkins Bloomberg School of Public Health has estimated that Lyme disease costs the US health care system an average of between $712m to $1.3 b annually, with costs increasing from $100 to almost $3000 per patient for managing early and late Lyme disease, respectively.

Diagnosis of Lyme disease other than by the characteristic bulls-eye rash is challenging. Though direct diagnostics methods for infectious diseases have advanced significantly, none are suitable for confirmation of Lyme disease due to the dissemination of the spirochete from the site of infection. Hence, PCR, culture, and antigen detection have poor sensitivity in the early stages of infection. Serology is the primary means of diagnosis for Lyme disease. However, the timing of a serological test and assay quality affect accurate diagnosis of Lyme disease, leading to a “window period” in the first 1-4 weeks post-acute infection when serological responses are limited.

In an attempt to increase the effectiveness of serological testing, the CDC introduced the standard two tier testing algorithm (STTA) in 1995, in which, the first tier is a sensitive Enzyme-Linked Immunosorbent Assay (ELISA) based detection of mostly IgG antibodies which, when positive or equivocal, is confirmed by a second-tier immunoblot to identify specific IgG and IgM antibodies to a panel of antigens that include Outer surface protein C (OspC), Borrelia membrane protein A (BmpA), flagellin (Fla), and GroEL (Centers for Disease Control and Prevention (CDC). Recommendations for test performance and interpretation from the Second National Conference on Serologic Diagnosis of Lyme Disease. MMWR Morb Mortal Wkly Rep. 1995 Aug. 11; 44(31):590-1). This protocol has drawbacks and is not suitable for detecting Lyme disease in the early acute phase because a robust titer of IgG antibodies is only detectable after 6 weeks of infection.

Existing ELISAs may not be suitable for detecting early IgM antibodies, while an immunoblot is cumbersome to perform and is prone to yield subjective interpretation of faint bands. To improve the efficacy of the algorithm, the CDC recently approved a modified two-tier testing algorithm (MTTA), which uses FDA cleared ELISAs for both tiers (Mead P, Petersen J, Hinckley A. Updated CDC Recommendation for Serologic Diagnosis of Lyme Disease. MMWR Morb Mortal Wkly Rep. 2019 Aug. 16; 68(32):703). Though advantageous in that objective interpretation is possible in an ELISA and the cumbersome immunoblot is replaced, the MTTA cannot show the detailed immune response to multiple antigens that the STTA offers (Marques A R. Revisiting the Lyme Disease Serodiagnostic Algorithm: the Momentum Gathers. Carroll K C, editor. J Clin Microbiol. 2018 August; 56(8):e00749-18).

### SUMMARY

The present disclosure provides a Lyme disease assay method comprising: conducting a first immunoassay on a patient blood, serum, or plasma sample in which a primary antibody in the sample binds to at least one individual spot of an array of spots each containing a separate antigen of a plurality of antigens associated with Lyme disease, and a secondary antibody binds to human IgG; conducting a second immunoassay on the sample in which the primary antibody binds to at least one individual spot of an array of spots identical to that of the first assay, and a secondary antibody binds to human IgM; detecting a plurality of signals from the first immunoassay and second immunoassay using image auto-analysis, each signal corresponding to a spot in the first immunoassay or the second immunoassay; analyzing the plurality of signals using a machine learning (ML)-assisted algorithm to determine if the patient sample is positive for Lyme disease.

The assay may further include the following features, which may be combined with one another and any other aspects of the present disclosure:
the immunoassay is an enzyme-linked immunosorbent assay (ELISA).
a likelihood of positive classification for Lyme disease is determined.
the determination of whether the patient sample is positive for Lyme disease and/or the likelihood of positive classification for Lyme disease are reported to a user.
the plurality of antigens comprise or consist of at least two, at least three, at least four, at least five, at least six, at least seven, at least eight, at least nine, at least ten or all eleven of VlsE, VoVo, VO4, VoBop, OspC-A, OspC-B, OspC-K, OspC-N, BmpA, DbpA, and DbpB.
at least one of the plurality of antigens may comprises or consists of a VoVo.
at least one of the plurality of antigens comprises or consists of a VO4.
at least one of the plurality of antigens comprises or consists of a VoBop.
at least one of the plurality of antigens comprises or consists of an OspC, further optionally wherein the OspC comprises a OspC-A, OspC-B, OspC-K, or OspC-N, or any combinations thereof, and still further optionally wherein the plurality of antigens comprises or consists of at least two, at least three, or all four of OspC-A, OspC-B, OspC-K, and OspC-N.
wherein at least one of the plurality of antigens comprises or consists of BmpA.
wherein at least one of the plurality of antigens comprises or consists of Decorin binding protein A (DbpA).
wherein at least one of the plurality of antigens comprises or consists of Decorin binding protein B (DbpB).
the plurality of antigens comprises or consists of at least two, at least three, or all four of i) at least one of VlsE, VoVo, or VO4; ii) at least one of VoVo, VO4, OspC-A, OspC-B, OspC-K, or OspC-N, iii) BmpA, and iv) at least one of DbpA or DbpB.
the plurality of antigens comprises or consists of at least one of VlsE, VoVo, or VO4 and at least one of VoVo, VO4, OspC-A, OspC-B, OspC-K, or OspC-N.
the plurality of antigens comprises or consists of at least one of DbpA or DbpB.
the ML-assisted algorithm has been trained using a random walk process.
the assay has a sensitivity of at least 40% for acute phase Lyme disease.
the assay has a specificity of at least 40% for post-acute phase Lyme disease.
the assay has a sensitivity of at least 90% for post-acute phase Lyme disease.
the assay has a specificity of at least 90% for post-acute phase Lyme disease.

The disclosure further provides a kit comprising an array of Lyme disease antigens and instructions for use according to any one of the above methods or other methods described herein.

The disclosure further provides a system comprising an array of Lyme disease antigens operable for use according to any one of the above methods or other methods described herein.

### BRIEF DESCRIPTION OF THE DRAWINGS

The present disclosure may be further understood though reference to the attached figures, in which:

FIG. 1 is a raw image (left panel) and an auto-analyzed image (right panel) of an array after an ELISA according to an assay of the present disclosure;

FIG. 2 is an example likelihood of a positive classification for past B. burgdorferi exposure indicator;

FIG. 3 is an example array layout as explained further in Example 1, in which 1 indicates anti-human IgG, 2 indicates bovine serum albumin (BSA), 3 indicates anti-human IgM, 4 indicates VoVo, 5 indicates VO4, 6 indicates VoBop, 7 indicates OspC-A, 8 indicates OspC-B, 9 indicates OspC-K, 10 OspC-N, 11 indicates VLSE, 12 indicates BmpA, 13 indicates DbpA, and 14 indicates DbpB;

FIG. 4 is an example set of raw images for a Lyme positive sample, with the IgG assay in the left panel and the IgM assay in the right panel;

FIG. 5 is an example user output showing an auto-analyzed image and allowing user adjustment;

FIG. 6 is a heatmap of antigen/control reactivity of known Lyme positive and negative samples as tested in Example 2;

FIG. 7 is a graph of the positive probability test results in one assay of Example 2, in which the right most dashed line indicates the threshold for positive status and the left-most dashed line indicates the threshold for negative status, with borderline status failing between the dashed lines;

FIG. 8 is an example decision tree for ML-assisted analysis.

### DETAILED DESCRIPTION

The present disclosure provides microarray assays that can detect IgM and IgG antibodies against a plurality of antigens associated with Lyme disease. Detection of a broad antigen response is clinically useful to understand the stage of Lyme disease and interpret symptoms. The present disclosure also provides machine learning (ML)-assisted methods to determine if IgG and IgM binding to the plurality of antigens is indicative of Lyme disease.

Assays of the present disclosure may have the ability to detect Lyme disease at the acute infection and post-acute infection stages.

Assays of the present disclosure may use a multiplexed immunoassay (e.g., ELISA) to measure the specific binding of IgG and IgM to each separate antigen relative to the concentration of IgG and IgM in the sample using automated image analysis. The assays may provide a quantitative readout.

Assays of the present disclosure may have a sensitivity at the acute and/or post-acute stages of Lyme disease at least equal to that of the CDC-recommended ELISA/immunoblot and/or ELISA/ELISA assays at the same stage of disease.

As used herein, Lyme disease is correlated with previous exposure to and/or infection with B. burgdorferi and, in some embodiments, may correlate with previous exposure to and/or infection with other BoreIlia species (such as Borrelia mayonii) or strains of B. burgdorferi prevalent in the location where the patient is domiciled, has been domiciled in the past, or has visited, or where the assay is conducted.

Acute Lyme disease occurs between three and thirty days after the patient being bitten by an infected tick and is characterized by one or more of the following symptoms:
Fever, chills, headache, fatigue, muscle and joint aches, and swollen lymph nodes, which may occur in the absence of rash.
Erythema migrans (EM) rash (in approximately 70 to 80 percent of infected persons).

EM may begin at the site of a tick bite after a delay of 3 to 30 days (about 7 days on average) and the rash expands gradually over several days reaching up to 12 inches or more (30 cm) across. The rash may feel warm to the touch but is rarely itchy or painful. The rash sometimes clears as it enlarges, resulting in a target or “bull's-eye” appearance. EM may appear on any area of the body and does not always appear as a “classic” EM rash.

Post-acute Lyme disease occurs days to months after tick bite and after resolution of the acute phase and is characterized by one or more of the following symptoms:
Severe headaches and neck stiffness
Additional EM rashes on other areas of the body
Facial palsy (loss of muscle tone or droop on one or both sides of the face)
Arthritis with severe joint pain and swelling, particularly the knees and other large joints.
Intermittent pain in tendons, muscles, joints, and bones
Heart palpitations or an irregular heart beat (Lyme carditis)
Episodes of dizziness or shortness of breath
Inflammation of the brain and spinal cord
Nerve pain
Shooting pains, numbness, or tingling in the hands or feet.

As used herein, “antibodies” is meant in a broad sense and includes immunoglobulin molecules belonging to any class. IgM antibodies are a specific class formed early during the immune response, while IgG antibodies are a separate class formed later in the immune response and may include the sub-classes IgG1, IgG2, IgG3 and IgG4. An antibody specifically binds to a given epitope on an antigen. IgG antibodies may have the same binding domains and bind to the same epitope as IgM antibodies present earlier during Lyme infection in the same patient. A patient may have IgG and IgM antibodies with the same binding domains that bind the same epitope present in the blood at the same time, or may have only IgG or IgM, depending on the stage of infection. Also, a patient may have antibodies with different binding domains that nevertheless specifically bind to the same antigen associated with Lyme disease, or even the same epitope on that antigen. Assays of the present disclosure may be able to detect any antibody that binds to a given antigen among the plurality of antigens associated with Lyme disease.

“Epitope” refers to a portion of an antigen to which an antibody specifically binds. Epitopes usually consist of chemically active (such as polar, non-polar or hydrophobic) surface groupings of moieties such as amino acids or polysaccharide side chains and may have specific three-dimensional structural characteristics, as well as specific charge characteristics. An epitope may be composed of contiguous and/or discontiguous amino acids that form a conformational spatial unit. For a discontiguous epitope, amino acids from differing portions of the linear sequence of the antigen come in close proximity in 3-dimensional space through the folding of the protein molecule. In some embodiments, antigens used in the present assay may be prepared and bound to a substrate such that even discontinuous epitopes are preserved.

“Specifically binds,” “specific binding” or “binds” refers to antibody binding to an antigen or an epitope within the antigen with greater affinity than for other antigens or epitopes. Typically, the antibody binds to the antigen or the epitope within the antigen with an equilibrium dissociation constant (K_{D}) of 1×10⁻⁷ M or less, for example 1×10⁻⁸ M or less, 1×10⁻⁹ M or less, 1×10⁻¹⁰ M or less, 1×10⁻¹¹ M or less, or 1×10⁻¹² M or less typically with a K_{D} that is at least one hundred-fold less than its K_{D} for binding to a non-specific antigen (e.g., BSA, casein). The K_{D} may be measured using standard procedures. Antibodies that specifically bind to the antigen or the epitope within the antigen may, however, have cross-reactivity to other related antigens.

Assays of the present disclosure may detect IgG and IgM antibodies that react to eleven antigens associated with Lyme disease. Arrays of the present disclosure may also detect IgG and IgM antibodies that react to at least two, at least three, at least four, at least five, at least six, at least seven, at least eight, at least nine, at least ten, at least eleven, at least twelve, at least fifteen, at least twenty, in a range between two and twenty, two and fifteen, two and twelve, two and eleven, two and ten, two and nine, two and eight, two and seven, two and six, two and five, two and four, two and three, three and twenty, three and fifteen, three and eleven, three and ten, three and five, five and twenty, five an fifteen, five and eleven, five and ten, eight and twenty, eight and fifteen, eight and eleven, eight and ten, ten and twenty, ten and fifteen, ten and eleven, eleven and twenty, or eleven and fifteen antigens associated with Lyme diseases.

In some embodiments, an antigen array is provided in which each of the plurality of antigens associated with Lyme disease are be bound to a substrate in such a manner as to allow specific binding of antibodies to such antigens in an immunoassay, such as an ELISA. Each antigen is bound to a different area of the substrate, typically in a known location or pattern with respect to the other antigens, allowing automated image analysis of the substrate to detect a signal that correlates with antibody binding to the antigen. Replicates may be included in the assay. In some embodiments, the antigens may be arranged in an a 6×6 antigen spot array containing duplicate spots for each of eleven antigens associated with Lyme disease, as illustrated in FIG. 3.

Antigens associated with Lyme disease used in assays of the present disclosure may be isolated bacterial peptide and/or recombinant antigens. In some embodiments, both the bacterial peptide and recombinant forms of an antigen may be included in the assay.

Antigens associated with Lyme disease may vary based on location, as different Borrellia are predominant in different regions. In the U.S., Borrelia burgdorferi s. stricto is the predominant Lyme-disease causing species, so antigens from this species may be used in assays for U.S. patients. Different antigens may be used for assays for patients in Europe or other locations. In some embodiments, the same antigens may be used to detect likely past exposure to any of multiple Borrelia species due to similarity in antigens across species and resulting cross-reactivity of antibodies.

In some embodiments, at least one of the plurality of antigens may be containing variable major protein (VMP)-like sequence (Vls) E lipoprotein (VlsE), which contains a C6 peptide domain. This lipoprotein undergoes antigenic variation in causes a strong IgG response.

In some embodiments, at least one of the plurality of antigens may be VoVo, which is a recombinant antigenic repeat sequence VlsE-OspC-VlsE-OspC. VoVo may provide a highly sensitive and specific target for both IgG and IgM.

In some embodiments, at least one of the plurality of antigens may be VO4, which is a recombinant protein in which VoVo is repeated four times. VO4 is also a target for both IgG and IgM.

In some embodiments, at least one of the plurality of antigens may be VoBop, such as BBK07 and oligopeptide permease (OppA2) which serves as a target for both early and late stage reactivity.

In some embodiments, at least one of the plurality of antigens may be an OspC, such as OspC-A, OspC-B, OspC-K, or OspC-N. In some embodiments, variants of the OspC may be included in the plurality of antigens to enhance IgM detection during acute Lyme disease because OspC is an early-stage immunogen. In particular, at least two, at least three, or all four of OspC-A, OspC-B, OspC-K, and OspC-N may be included in the plurality of antigens.

In some embodiments, at least one of the plurality of antigens may be BmpA.

In some embodiments, at least one of the plurality of antigens may be Decorin binding protein A (DbpA).

In some embodiments, at least one of the plurality of antigens may be Decorin binding protein B (DbpB).

In some embodiments, the plurality of antigens may include at least two, at least three, at least four, at least five, at least six, at least seven, at least eight, at least nine, ten or all eleven of VlsE, VoVo, VO4, VoBop, OspC-A, OspC-B, OspC-K, OspC-N, BmpA, DbpA and DbpB.

In some embodiments, the plurality of antigens may include at least two, at least three, or all four of i) at least one of VlsE, VoVo, or VO4; ii) at least one of VoVo, VO4, OspC-A, OspC-B, OspC-K, or OspC-N, iii) BmpA, and iv) at least one of DbpA or DbpB.

In some embodiments, the plurality of antigens may include at least one of VlsE, VoVo, or VO4 and at least one of VoVo, VO4, OspC-A, OspC-B, OspC-K, or OspC-N.

In some embodiments, the plurality of antigens may include at least one of DbpA or DbpB.

In some embodiments, the plurality of antigens and the IgG and IgM controls are the same in the array for IgM detection and the array for IgG detection. In some embodiments, they may be in same patterns in the array for IgM detection and the array for IgG detection. This may help avoid assay errors by not requiring the addition of a specific secondary antibody to a specific well or other substrate.

In some embodiments, the array is a microarray, e.g and array in which each antigen spot has an average surface dimension, such as an average diameter, in a range between 1-500 μm 1-300 μm, 1-1000 μm, 1-999 μm, 100-1000 μm, 100-500 μm, 100-300 μm, 200-400 μm, 200-500 μm, or 300-500 μm.

The substrate may be any substrate compatible with an immunoassay, such as an ELISA, but, in many embodiments, it will be a commonly used ELISA vessel, such as a microtiter plate, particularly a 96-well microtiter plate. In order to allow smaller numbers of assays to be run, coated microtiter plate wells may be provided in strips, for example of 8 wells per strip for a 96-well microtiter plate assay, that may be placed in a standard size plate along with other strips, blanks, or unoccupied space. The arrays also include IgG and/or IgM capture controls. In some embodiments, these capture controls may be anti-human IgG and anti-human IgM antibodies. In some embodiments, the IgG and/or IgM capture controls may be in the corners of the array to help frame the array layout for the automated image analysis.

For each sample, at least two antigen arrays may be used, one to detect IgG and one to detect IgM.

In some embodiments of the present disclosure, the sample may be blood, serum, plasma, or cerebral spinal fluid from a patient.

In the ELISA, a sample is contacted with at least two antigen arrays under conditions sufficient to allow specific binding of antibodies to each of the antigens, if present in the sample. Typically the blood sample is diluted prior to contact with the antigen array. The sample may be fresh, or it may be stored in a manner that preserves antibodies, such as by dilution and processing or by freezing. In some embodiments, treatment of the sample may be indicated in the ML model, which may use this information in determining Lyme positive probability in the patient who provided the sample. For instance, lower levels of antibodies may be considered sufficient to presume a patient positive for Lyme disease if the sample was treated in a manner that causes increased antibody degradation as compared to other treatments.

Next, a secondary antibody reactive to IgG is added to one antigen array, while a secondary antibody reactive to IgM is added to the second antigen array, both under conditions to allow specific binding to IgG or IgM in the sample. These secondary antibodies typically include a tag to allow their detection. Any such tag commonly used in ELISAs is appropriate. For example, a fluorescent, chemiluminescent or enzymatic tag may be used. If needed, any detection reagent necessary to render the tag detectable, such as an enzymatic substrate, is then added. Then the array is imaged.

The array is aspirated or washed at least before imaging to remove unbound tagged secondary antibody. Typically the array is also washed between binding of antibodies in the sample to antigens and addition of the secondary antibody and, if a detection reagent is added, between binding of the secondary antibody and addition of the detection reagent.

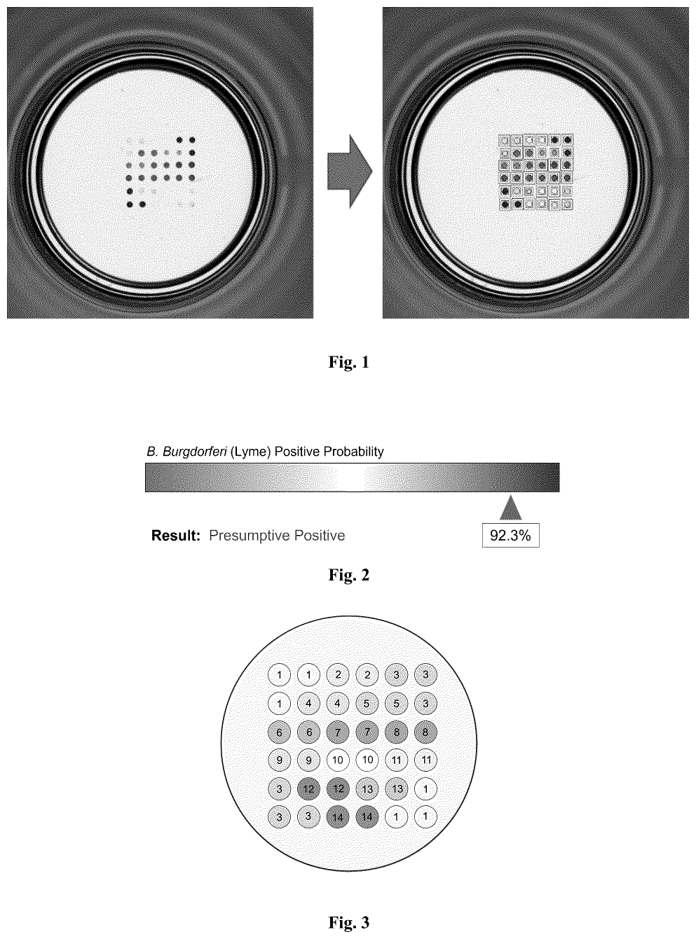

Imaging results in a raw image, as illustrated in the left array image of FIG. 1. The secondary antibody tag or a product of its reaction with a detection reagent produces a raw signal in the raw image for each antigen that corresponds to the amount of antibody in the sample that specifically binds that antigen.

Next, an automated image analyzer detects the antigen spots and an adjacent portion of the substrate, as illustrated in the right array image of FIG. 1. The results of this detection may be superimposed on an image of the array to create an auto-analyzed image, such that a user can verify that the antigen spots and adjacent portions have been correctly identified. In some embodiments, the user may adjust the positions of the antigen spots and adjacent portions to place them in more accurate locations in the auto-analyzed image.

Next, the auto-analyzed image, as position-adjusted by the user in some embodiments, is analyzed to determine a raw signal for each antigen spot in the array. In some embodiments, the raw signal is further processed into an analysis signal to increase accuracy. For example, a raw signal for the adjacent portion of the array may be determined and subtracted from the raw signal of the antigen spot. This removes background signal not related to antibody binding from the analysis signal. In other embodiments, the top and bottom 25% of the signal values for points within the antigen spot and/or the adjacent portion of the substrate may be discarded. This helps eliminate the effects of contaminants, such as a hair or other foreign material that is unequally distributed on the array on the analysis signal.

The raw signal and/or the analysis signal may also be compared to the signal value for an IgG control and/or IgM control in the same array. Typically the raw signal and/or analysis signal in an ELISA where the secondary antibody binds IgG is compared to the IgG control and the signal for the ELISA where the secondary antibody binds IgM is compared to the IgM control. Comparison with the controls allows the generation of a proportional signal for each antigen spot on the array, which reflects the amount of IgG or IgM bound to the antigen as a proportion of the total IgG or IgM. The proportional signal may reflect the stage of Lyme infection. The proportional signal may also help avoid false negatives due to low total IgG or IgM levels.

Next the raw signals, analysis signals and/or proportional signals are provided to an ML-assisted auto-analyzer trained to detect Lyme disease based on signals from the ELISAs performed. The auto-analyzer uses this information to provide a positive or negative result for Lyme disease. A likelihood of a positive classification for past B. burgdorferi exposure may also be indicated, as shown in FIG. 2. This information may then be provided to a user. In some embodiments, a simple positive or negative results may be provided in addition to or in place of the likelihood of positive classification.

In general, when analyzing a sample using a forest-based ML model, the ML model implements a given number of decision trees, each of which determines whether the sample is classified as positive or negative based on the parameters and structure of that decision tree. For most samples, some decision trees will result in a positive determination, while others will result in a negative determination. The pool of classification results from the decision trees forms the “forest.”

The classifications from all decision trees (or, in some more complex models, a subset of the decision trees) are then combined to determine whether the sample is positive or negative for Lyme disease.

In some embodiments, the positive or negative status may be a simple binary indication based on whether a threshold number of decision trees found the sample to be positive. For example, if fifty percent or more of the total number of decision trees found the sample to be positive, then the sample may be reported as positive. If greater certainty is required then the threshold may be higher, such as sixty, seventy, eighty, or ninety percent of the total number of decision trees.

In other embodiments, a likelihood of a positive classification is determined that reflects the proportion of decision trees in the ML model that classified the sample as positive for Lyme disease. For instance, if the ML model implements 1000 decision trees and 889 of those decision trees classify a given sample as positive, then the likelihood of a positive classification is 88.9%.

In some embodiments, an image such as the raw image or auto-analyzed image, may also be provided to the user to allow independent visual verification of the positive or negative result. For example, if the user sees a large number of dark spots in the raw or auto-analyzed image, but the overall test result is negative, or if the user sees almost no dark spots in the raw or auto-analyzed image, but the overall test result is positive, the user may doubt the result and re-run the detection, auto-analysis, or even the entire assay with the same or a different sample from the patient. In some embodiments, the user may be instructed to confirm using visual verification of results within a certain likelihood of positive classification range, such as between 20% and 80%.

In some embodiments, the sample may be collected from a patient more than seven days after infection with B. burgdorferi.

In some embodiments, the assay may have a sensitivity and/or a specificity of at least 40%, at least 45% at least 50%, at least 60%, or in a range between 40% and 70%, 40% and 60%, 40% and 50%, 45% and 70%, 45% and 60%, 45% and 50%, 50% and 70%, or 50% and 60% during the acute phase of infection.

In some embodiments, the assay may have a sensitivity and/or a specificity of at least 80%, at least 85%, at least 90%, at least 95%, or in a range between 80% and 100%, 80% and 99%, 80% and 95%, 80% and 90%, 80% and 85%, 85% and 100%, 85% and 99%, 85% and 95%, 85% and 90%, 90% and 100%, 90% and 99%, 90% and 95%, 95% and 100%, or 95% and 99% during the post-acute phase of infection.

In some embodiments, the assay may have an accuracy that is determined by disease prevalence and any of the above-noted specificities and/or sensitivities; accuracy is the (sensitivity×prevalence)+(specificity×(1-prevalence)). Accuracy may also be determined for the acute and post-acute phases of infection.

In some embodiments, the assay may provide separate results for IgG detection, for IgM detection assay, or both. Such results may be provided in addition to results as discussed above using detection of both IgG and IgM, typically by at least two antigen arrays.

IgM results may be particularly useful in indicating whether a patient is in the acute phase of infection, or if a patient previously infected has been re-exposed. Patients who have been re-exposed may have previously exhibited higher IgG activity as compared to IgM activity, may exhibit an increase in IgM activity, or may have either or both IgG and IgM activity above a certain threshold.

The ML-assisted auto-analyzer may be trained by providing images of arrays run with samples from known positive and negative patients at different stages of Lyme detection. The ML model may be set to assign a positive or negative value to a sample based on the analysis signals for each of the antigens as compared to the analysis signals for total IgG and/or total IgM for the array. This assigned value is then compared to the true positive or negative value of the sample and, if not accurate, the algorithm adjusts using any number of AI or ML-training processes, such as random walks, particularly a random forest classifier (RFC), and reassigns values using the adjusted algorithm. Once a set specificity and/or sensitivity, or any other accuracy value has been reached, typically when the algorithm is used to evaluate a set of known samples other than the training set, the algorithm is deemed trained and may be used in an assay as disclosed herein.

In some embodiments, the assay may also detect and the ML-assisted auto-analyzer may be trained for common co-infections of Lyme disease, such as Babesia microti, which causes Babesiosis, by including at least one antigen associated with the co-infective organism.

The disclosure provides both methods of conducting assays as disclosed herein and systems or kits operable of use in conducting such assays. Systems and kits may include assay materials, such as a substrate with at least two arrays as described herein. Other reagents, such as sample preparation materials, secondary antibodies, detection reagents, and wash/aspiration buffers may also be provided in systems and kits, as may instructions for use. Systems may further include detection devices and/or a processor operable to implement the ML methods and/or provide an assay result to a user.

Elements of the different embodiments disclosed herein, including those in the Examples, may be combined with one another, Furthermore, not all aspects described in connection with a given embodiment are required for that embodiment to function and many aspects are provided merely as details to assist in implementing the disclosure.

### EXAMPLES

### Example 1—Assay Development

A library of relevant B. burgdorferi antigens were screened for seroreactivity against confirmed Lyme positive samples. These antigens are: VoVo, VO4, VoBop, OspC-A, OspC-B, OspC-K, OspC-N, VlsE, BmpA, DbpA and DbpB.

To properly approach the early serodiagnosis of samples from patients who may have Lyme disease, each target antigen was individually evaluated by using a microarray on a standard microtiter plate well of a 96-well plate. A 6×6 array of antigens was spotted on the bottom of each test well. This allowed for multiple replicates of control reagents (e.g., anti-Hu IgG, anti-Hu IgM and BSA) and duplicate spots for each of the B. burgdorferi antigens.

A standard ELISA protocol was then performed and each diluted sample was tested in two separate ELISA plate wells—one well for IgG reactivity and the other for IgM reactivity. A single ELISA plate could be used to screen 48 samples (including Positive and Negative controls), permitting high throughput screening options in laboratory-based settings.

As there were 6×6=36 total spots in each well, every sample provided a total of 72 data points (36 for IgG and 36 for IgM) corresponding to raw signals and/or analyzed signals. These data points may be appropriately averaged for spots representing the same target antigen and the within-well standard deviations may be automatically calculated. Spot mapping is shown in FIG. 3. Control spots of anti-human IgG and anti-human IgM were replicated in the corners of the array. These corner spots helped frame the array layout.

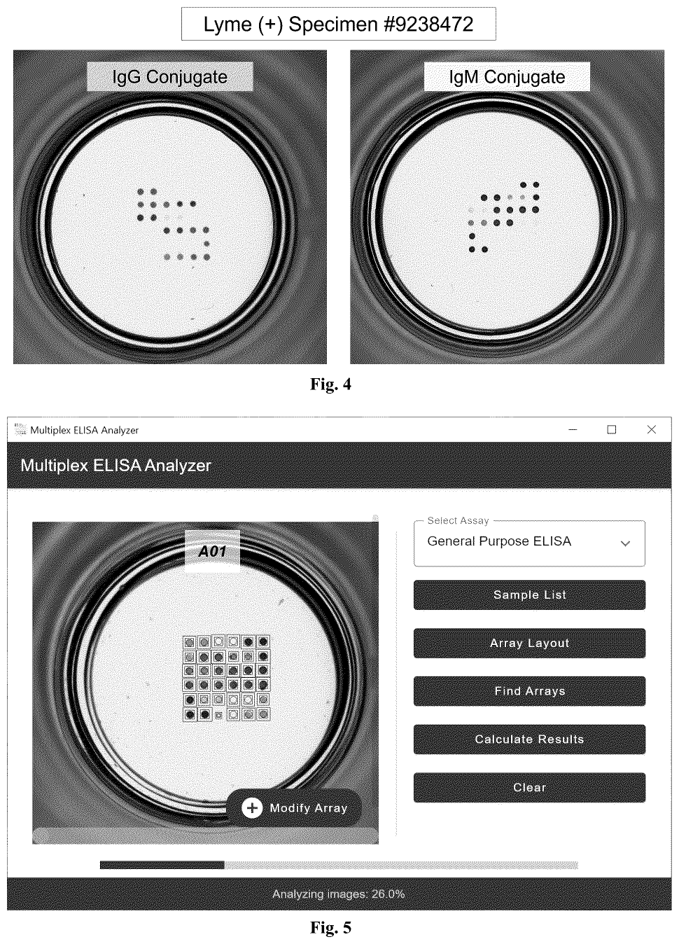

An example set of raw images for a Lyme positive sample is shown in FIG. 4, with the IgG assay in the left panel and the IgM assay in the right panel. As can be seen, the IgG and IgM conjugate control spots provided strong internal positive controls at the appropriate locations. Additionally, a variety of target antigens were reactive with varying signal strengths. Once the ELISA plate images were recorded, a critical analytical tool was used to properly quantify the raw signal, analyzed signal, and/or proportional signal for each of the individual test spots and to determine whether the sample was positive or negative for Lyme disease. To this end, a custom software solution was created using Python. The software automatically locates the array locations and individual test spots and permits the operator to view all the raw and auto-analyzed image, for example as shown in FIG. 5. The software solution robustly located the test spots when provided with the array format of FIG. 3.

### Example 2—Assay Assessment

To evaluate the current performance of the assay developed in Example 1, a total of 182 samples, including 100 non-endemic normal human serum samples, 7 rheumatoid factor (RF) positive samples, 5 human anti-mouse antibody (HAMA) positive samples, 24 commercially acquired Lyme positive samples, CDC Lyme Panel #1 (which had 5 Lyme positive and 5 Lyme negative samples), and CDC Lyme Panel #2 (which had 12 Lyme positive and 20 Lyme negative samples) were tested. Of note, CDC panel members included samples categorized as “Acute Lyme disease”, some of which were considered two-tier test negative.

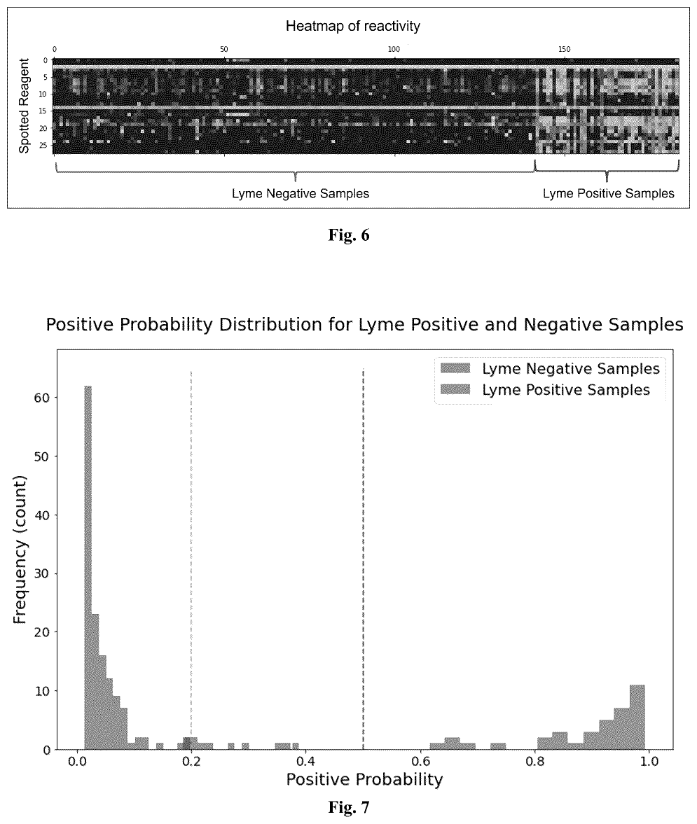

A heatmap for each sample reactivity (IgG and IgM) against each of the spotted antigens/controls is shown in FIG. 6. As can be seen, a significant but varying signal strength was present in the confirmed Lyme positive sample set with a variety of antigens associated with Lyme disease; however, there are varying background level signals with these antigens among the presumptive Lyme negative samples, indicating that developing a simple threshold of values of positive or negative for each antigen may be insufficient, hence the need for an ML-assisted analysis.

Instead of approaching the analysis by attempting to establish manual cut-off values (e.g., using receiver operating characteristic (ROC) curve analyses, etc.) for each individual antigen associated with Lyme disease, a random forest classifier (RFC) model was used to better incorporate the complexity of the immune response to the individual targets.

A random forest classifier (RFC) model was generated using n=1000 estimators. The RFC model uses averaging over the estimators to improve the predictive accuracy. Furthermore, a 10-fold cross-validation analysis (using a test size of 30% of the total dataset) was employed to help estimate the accuracy and evaluate the robustness of the generated models. In particular, the RFC models were 10-fold cross-validated using a randomized subset of the complete dataset. 30% of the data (n=56 samples) are randomly selected to be part of the “validation” testing while the remaining 70% of the samples were used for the RFC training process. After training, the validation dataset was evaluated for accuracy, specificity and sensitivity. The results for each fold validation are shown in Table 1. Specificity was maintained at 100% throughout while sensitivity ranged from 81.82%-100.0%., depending upon which set of positive samples (i.e., problematic acute samples) were included with the validation sample set. The overall test accuracy ranged from 94.64%-100%. The positive samples that were not detected were from acute samples that also tested as negative using a standard two-tier test.

TABLE 1

| Fold | | | |
|---|---|---|---|
| Evaluation | Accuracy | Specificity | Sensitivity |
| 1 | 98.21% | 100% (45/45) | 90.91% (10/11) |
| 2 | 96.43% | 100.00% (44/44) | 83.33% (10/12) |
| 3 | 94.64% | 100.00% (38/38) | 83.33% (15/18) |
| 4 | 96.43% | 100.00% (43/43) | 84.62% (11/13) |
| 5 | 98.21% | 100.00% (46/46) | 90.00% (9/10) |
| 6 | 100.00% | 100.00% (47/47) | 100.00% (9/9) |
| 7 | 96.43% | 100.00% (46/46) | 80.00% (8/10) |
| 8 | 96.43% | 100.00% (41/41) | 86.67% (13/15) |
| 9 | 98.21% | 100.00% (47/47) | 88.89% (8/9) |
| 10 | 96.43% | 100.00% (45/45) | 81.82% (9/11) |

Using one of the random forest classifier models trained on a subset of the complete dataset, the positive percent confidence value for all the tested samples was determined. Any sample with a confidence value ≥50% was considered positive for Lyme disease. Samples with a confidence value ranging from 20%-50% were currently considered borderline. Of note, only 4 out of 141 Lyme presumptive negative samples were categorized as borderline (2.84%). None of the Lyme negative samples were categorized as positive. A summary table of the test results is shown in Table 2 and a distribution of the positive probability test results is shown in FIG. 7.

TABLE 2

| | | Presumptive Truth | |
|---|---|---|---|
| | | Negative | Positive |
| Test Assay | Negative | 137 | 0 |
| | Borderline | 4 | 4 |
| | Positive | 0 | 37 |

Of the Lyme positive samples, five were categorized by the CDC as acute and negative under the current two-tier test. Of these samples, 40% (2/5) were categorized as positive using the assay and RFC model interpretation of this example. The remaining three samples were categorized as borderline. These results are summarized in Table 3.

TABLE 3

| | Test Assay: | | Lyme | Ref. | Ref. | IgM | IgM | |
|---|---|---|---|---|---|---|---|---|
| | Positive | Test Assay: | Acute/ | EIA | EIA | WB | WB | 2-Tier |
| Sample ID | Probability | Interpretation | Convalescent | Value | Interp. | Interp. | Bands | Interp. |
| Panel #1 - 9552 | 33.51% | Borderline | Acute | 0.25 | Neg | Neg | None | Neg |
| Panel #2 - 2367 | 22.26% | Borderline | Acute | 0.07 | Neg | Neg | | Neg |
| Panel #2 - 8130 | 21.66% | Borderline | Acute | 0.39 | Neg | Neg | | Neg |
| Panel #2 - 4232 | 61.66% | Positive | Acute | 0.11 | Neg | Neg | — | Neg |
| Panel #2 - 6794 | 66.90% | Positive | Acute | 0.72 | Neg | Pos | 41, 23 | Neg |

Additionally, four Lyme negative samples provided as part of CDC Panel #2 were considered “problematic” with the current ELISA and were considered current ELISA false positive. All four of these problematic samples were categorized as negative using the assay and RFC model interpretation of this example. The results are summarized in Table 4.

TABLE 4

| | Test Assay: | | | | |
|---|---|---|---|---|---|
| | Positive | Test Assay: | | EIA | EIA |
| Sample ID | Probability | Interpretation | Sample Group | Value | Interpretation |
| Panel #2-1435 | 17.31% | Negative | Syphilis | 1.73 | Pos |
| Panel #2-5406 | 5.85% | Negative | Healthy endemic | 1.29 | Pos |
| Panel #2-6485 | 2.21% | Negative | Mononucleosis | 3.17 | Pos |
| Panel #2-4648 | 4.26% | Negative | Healthy non-endemic | 1.18 | Pos |

### Example 3—Random Forest Classifier Decision Tree

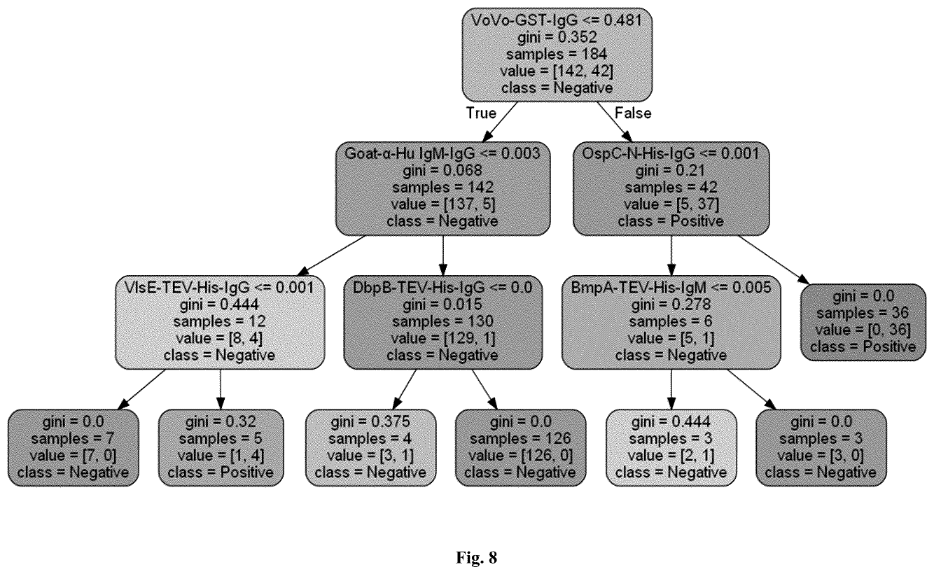

A single exemplary decision tree is shown in FIG. 8 indicating the logical path for determining a sample status (i.e., Lyme positive or negative) in the assay of Example 2. For instance, if a sample has a signal ratio for VoVo:IgG of >0.481 (reflecting the value of the IgG reactivity to the VoVo antigen divided by the anti-Hu IgG reactivity (total IgG) in the same well), it is initially considered positive. Following down the decision tree, if the signal ratio for OspC:IgG is >0.001, then the sample is classified as Lyme Positive. If the signal ratio is <=0.001, then the sample goes to another decision point with the BmpA:IgM ratio to evaluate another decision point. In a similar manner, all samples can be categorized by following this decision tree.

The random forest classifier approach used n=1000 individual decision trees and took the aggregate results for better classifying each specimen.

### Example 4—Assay Assessment Using CDC Panels

The assay of Example 1 was further tested using CDC Panels #1 and #2 which include 10 Early Lyme-EM positive specimens, Lyme arthritis, Neurologic Lyme and a mixture of negative control samples. Of the 10 Early Lyme-EM samples, 5 are positive by the standard two-tier testing algorithm (data provided by CDC). Of the same 10 samples, 9 out of 10 samples were considered either Borderline Reactive or Positive with the multiplex Lyme assay of Example 1. Four samples from CDC Panel #2 were out of volume and could not be included with this testing. This includes one nerologic Lyme sample and three negative control specimens. Results are summarized in Table 5 and underlying data is provided in Table 6.

TABLE 5

| | Early Lyme-EM | Lyme arthritis | Neurologic Lyme | Negative Controls |
|---|---|---|---|---|
| % Positive, Standard | 50% (5/10) | 100% (4/4) | 100% (2/2) | 0% (0/22) |
| Two-Tier Testing | | | | |
| % Positive, Multiplex | 90% (9/10) | 100% (4/4) | 100% (2/2) | 0% (0/22) |
| Lyme Assay | | | | |

TABLE 6

| | Positive | |
|---|---|---|
| Sample ID | Probability | Sample Interpretation |
| Positive Control | 100.00% | Presumptive Positive for Lyme Disease |
| Negative Control | 0.12% | Presumptive Negative for Lyme Disease |
| LV #01 | 57.74% | Presumptive Positive for Lyme Disease |
| LV #02 | 99.80% | Presumptive Positive for Lyme Disease |
| LV #03 | 100.00% | Presumptive Positive for Lyme Disease |
| LV #04 | 100.00% | Presumptive Positive for Lyme Disease |
| LV #05 | 100.00% | Presumptive Positive for Lyme Disease |
| LV #06 | 13.39% | Presumptive Negative for Lyme Disease |
| LV #07 | 13.83% | Presumptive Negative for Lyme Disease |
| LV #08 | 10.75% | Presumptive Negative for Lyme Disease |
| LV #09 | 13.65% | Presumptive Negative for Lyme Disease |
| LV #10 | 5.15% | Presumptive Negative for Lyme Disease |
| R39 #01 | 25.21% | Presumptive Negative for Lyme Disease |
| R39 #02 | 42.45% | Borderline Reactive for Lyme Disease |
| R39 #03 | 75.99% | Presumptive Positive for Lyme Disease |
| R39 #04 | 17.78% | Presumptive Negative for Lyme Disease |
| R39 #05 | 9.95% | Presumptive Negative for Lyme Disease |
| R39 #06 | 5.36% | Presumptive Negative for Lyme Disease |
| R39 #08 | 16.74% | Presumptive Negative for Lyme Disease |
| R39 #10 | 8.34% | Presumptive Negative for Lyme Disease |
| R39 #11 | 100.00% | Presumptive Positive for Lyme Disease |
| R39 #12 | 19.90% | Presumptive Negative for Lyme Disease |
| R39 #13 | 100.00% | Presumptive Positive for Lyme Disease |
| R39 #14 | 0.75% | Presumptive Negative for Lyme Disease |
| R39 #15 | 0.81% | Presumptive Negative for Lyme Disease |
| R39 #16 | 10.05% | Presumptive Negative for Lyme Disease |
| R39 #17 | 10.45% | Presumptive Negative for Lyme Disease |
| R39 #18 | 12.96% | Presumptive Negative for Lyme Disease |
| R39 #19 | 5.23% | Presumptive Negative for Lyme Disease |
| R39 #20 | 79.16% | Presumptive Positive for Lyme Disease |
| R39 #22 | 0.23% | Presumptive Negative for Lyme Disease |
| R39 #23 | 99.90% | Presumptive Positive for Lyme Disease |
| R39 #24 | 100.00% | Presumptive Positive for Lyme Disease |
| R39 #25 | 16.30% | Presumptive Negative for Lyme Disease |
| R39 #26 | 100.00% | Presumptive Positive for Lyme Disease |
| R39 #28 | 0.43% | Presumptive Negative for Lyme Disease |
| R39 #29 | 100.00% | Presumptive Positive for Lyme Disease |
| R39 #30 | 100.00% | Presumptive Positive for Lyme Disease |
| R39 #31 | 9.25% | Presumptive Negative for Lyme Disease |
| R39 #32 | 7.71% | Presumptive Negative for Lyme Disease |
| RD1146 #11 | 9.99% | Presumptive Negative for Lyme Disease |
| RD1146 #13 | 3.25% | Presumptive Negative for Lyme Disease |
| RD1146 #14 | 0.03% | Presumptive Negative for Lyme Disease |
| RD1146 #15 | 5.77% | Presumptive Negative for Lyme Disease |
| RD1146 #17 | 5.69% | Presumptive Negative for Lyme Disease |
| RD1146 #18 | 0.04% | Presumptive Negative for Lyme Disease |
| RD1146 #19 | 1.55% | Presumptive Negative for Lyme Disease |
| RD1146 #20 | 2.96% | Presumptive Negative for Lyme Disease |

### Example 5—Assay Assessment Using Lyme Disease Biobank Specimens

Lyme Disease Biobank (LDB) specimens that include 25 Early-Lyme (EM Positive) specimens were also tested using the multiplex Lyme assay of Example 1. Of the 25 samples, 12 are positive via the standard two-tier testing algorithm (data provided by LDB). Of the 25 samples, 21 were found Positive or Borderline Reactive with multiplex Lyme assay of Example 1. Results are summarized in Table 7 and underlying data is provided in Table 8.

TABLE 7

| | Early Lyme-EM Specimens |
|---|---|
| % Positive, Standard two-tier testing | 48% (12/25) |
| % Positive, Multiplex Lyme Assay | 84% (21/25) |

TABLE 8

| | Positive | |
|---|---|---|
| Sample ID | Probability | Sample Interpretation |
| LD104075 | 91.01% | Presumptive Positive for Lyme Disease |
| LD104076 | 38.76% | Borderline Reactive for Lyme Disease |
| LD104077 | 100.00% | Presumptive Positive for Lyme Disease |
| LD104078 | 99.90% | Presumptive Positive for Lyme Disease |
| LD104079 | 91.23% | Presumptive Positive for Lyme Disease |
| LD104080 | 96.50% | Presumptive Positive for Lyme Disease |
| LD104081 | 94.40% | Presumptive Positive for Lyme Disease |
| LD104082 | 98.39% | Presumptive Positive for Lyme Disease |
| LD104083 | 24.56% | Presumptive Negative for Lyme Disease |
| LD104084 | 64.64% | Presumptive Positive for Lyme Disease |
| LD104085 | 18.60% | Presumptive Negative for Lyme Disease |
| LD104086 | 61.14% | Presumptive Positive for Lyme Disease |
| LD104087 | 100.00% | Presumptive Positive for Lyme Disease |
| LD104088 | 71.36% | Presumptive Positive for Lyme Disease |
| LD104089 | 100.00% | Presumptive Positive for Lyme Disease |
| LD104090 | 43.58% | Borderline Reactive for Lyme Disease |
| LD104091 | 100.00% | Presumptive Positive for Lyme Disease |
| LD104092 | 100.00% | Presumptive Positive for Lyme Disease |
| LD104093 | 99.60% | Presumptive Positive for Lyme Disease |
| LD104094 | 55.13% | Presumptive Positive for Lyme Disease |
| LD104095 | 98.00% | Presumptive Positive for Lyme Disease |
| LD104096 | 47.25% | Borderline Reactive for Lyme Disease |
| LD104097 | 35.98% | Borderline Reactive for Lyme Disease |
| LD104098 | 27.65% | Presumptive Negative for Lyme Disease |
| LD104099 | 25.05% | Presumptive Negative for Lyme Disease |
| KB97201 | 6.26% | Presumptive Negative for Lyme Disease |
| KB97202 | 3.90% | Presumptive Negative for Lyme Disease |
| KB97203 | 8.12% | Presumptive Negative for Lyme Disease |
| KB97204 | 2.46% | Presumptive Negative for Lyme Disease |
| KB97205 | 3.66% | Presumptive Negative for Lyme Disease |
| KB97206 | 5.74% | Presumptive Negative for Lyme Disease |
| KB97207 | 17.53% | Presumptive Negative for Lyme Disease |
| KB97208 | 0.06% | Presumptive Negative for Lyme Disease |
| KB97209 | 2.31% | Presumptive Negative for Lyme Disease |
| KB97210 | 1.30% | Presumptive Negative for Lyme Disease |
| KB97211 | 0.47% | Presumptive Negative for Lyme Disease |
| KB97212 | 6.21% | Presumptive Negative for Lyme Disease |
| KB97213 | 7.15% | Presumptive Negative for Lyme Disease |
| KB97214 | 18.21% | Presumptive Negative for Lyme Disease |
| KB97215 | 3.40% | Presumptive Negative for Lyme Disease |
| KB97216 | 1.46% | Presumptive Negative for Lyme Disease |
| KB97217 | 1.12% | Presumptive Negative for Lyme Disease |
| KB97218 | 1.53% | Presumptive Negative for Lyme Disease |
| KB97219 | 2.08% | Presumptive Negative for Lyme Disease |
| KB97220 | 6.98% | Presumptive Negative for Lyme Disease |
| KB97221 | 28.57% | Presumptive Negative for Lyme Disease |

### Example 6—Assay Assessment For Early Lyme Specimens

Combining the Early Lyme-EM specimens from CDC Panels #1 and #2 and LDB results in a total of 35 Early Lyme-EM samples. Of these samples, 17 (48.6%) are positive with the standard two-tier testing algorithm. Of these same specimens, 30 (85.7%) were found to be positive or borderline reactive with multiplex assay of Example 1. Results are summarized in

Table 9 and underlying data is provided in Table 6 and Table 8.

TABLE 9

| | Early Lyme-EM Specimens |
|---|---|
| % Positive, Standard two-tier testing | 48.6% (17/35) |
| % Positive, Multiplex Lyme Assay | 85.7% (30/35 |

Accordingly, these results establish that the assay of the present disclosure is able to accurately detect Lyme disease in the early stages of infection.

The various embodiments described above can be combined to provide further embodiments. All of the U.S. patents, U.S. patent application publications, U.S. patent applications, foreign patents, foreign patent applications and non-patent publications referred to in this specification, are incorporated herein by reference, in their entirety. Aspects of the embodiments can be modified, if necessary to employ concepts of the various patents, applications and publications to provide yet further embodiments.

All numerical values provided herein, with the exception of numerical values reflecting actual data, include values “about” the recited value, such as or within +/−5% of the recited value.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A Lyme disease assay method comprising:
conducting a first immunoassay on a patient blood, serum, or plasma sample in which a primary antibody in the sample binds to at least one individual spot of an array of spots each containing a separate antigen of a plurality of antigens associated with Lyme disease, and a secondary antibody binds to human IgG;
conducting a second immunoassay on the sample in which the primary antibody binds to at least one individual spot of an array of spots identical to that of the first assay, and a secondary antibody binds to human IgM;
detecting a plurality of signals from the first immunoassay and second immunoassay using image auto-analysis, each signal corresponding to a spot in the first immunoassay or the second immunoassay;
analyzing the plurality of signals using a machine learning (ML)-assisted algorithm to determine if the patient sample is positive for Lyme disease.

2. The assay of claim 1, wherein the immunoassay is an enzyme-linked immunosorbent assay (ELISA).

3. The assay of claim 1, wherein a likelihood of positive classification for Lyme disease is determined.

4. The assay of claim 1, wherein the determination of whether the patient sample is positive for Lyme disease and/or the likelihood of positive classification for Lyme disease are reported to a user.

5. The assay of claim 1, wherein the plurality of antigens comprise at least two of VlsE, VoVo, VO4, VoBop, OspC-A, OspC-B, OspC-K, OspC-N, BmpA, DbpA, and DbpB.

6. The assay of claim 1, wherein at least one of the plurality of antigens comprises a VoVo.

7. The assay of claim 1, wherein at least one of the plurality of antigens comprises a VO4.

8. The assay of claim 1, wherein at least one of the plurality of antigens comprises a VoBop.

9. The assay of claim 1, wherein at least one of the plurality of antigens comprises an OspC.

10. The assay of claim 9, wherein the OspC comprises a OspC-A, OspC-B, OspC-K, or OspC-N, or any combinations thereof.

11. The assay of claim 1, wherein at least one of the plurality of antigens comprises a BmpA.

12. The assay of claim 1, wherein at least one of the plurality of antigens comprises a Decorin binding protein A (DbpA).

13. The assay of claim 1, wherein at least one of the plurality of antigens comprises a Decorin binding protein B (DbpB).

14. The assay of claim 1, wherein the plurality of antigens comprises at least two of i) at least one of VlsE, VoVo, or VO4; ii) at least one of VoVo, VO4, OspC-A, OspC-B, OspC-K, or OspC-N, iii) BmpA, and iv) at least one of DbpA or DbpB.

15. The assay of claim 1, wherein the plurality of antigens comprises or consists of at least one of DbpA or DbpB.

16. The assay of claim 1, wherein the assay has a sensitivity of at least 40% for acute phase Lyme disease.

17. The assay of claim 1, wherein the assay has a specificity of at least 40% for post-acute phase Lyme disease.

18. A kit comprising an array of Lyme disease antigens and instructions for use according to claim 1.

19. A system comprising an array of Lyme disease antigens operable for use according to claim 1.
